(19) United States
(12) Patent Application Publication
(10) Pub. No.: US 2014/0081218 A1
(43) Pub. Date: Mar. 20, 2014

(54) INTRAVENOUS DEVICE HAVING A MOVABLE ARRANGEMENT

(71) Applicants: Sidney J. Winawer, New York, NY (US); Paul Richard Booth, New York, NY (US)

(72) Inventors: Sidney J. Winawer, New York, NY (US); Paul Richard Booth, New York, NY (US)

(73) Assignee: MEMORIAL SLOAN-KETTERING CANCER CENTER, New York, NY (US)

(21) Appl. No.: 14/017,592

(22) Filed: Sep. 4, 2013

Related U.S. Application Data
(60) Provisional application No. 61/697,423, filed on Sep. 6, 2012.

(51) Int. Cl. A61M 5/168

(52) U.S. Cl. CPC A61M 5/16886
USPC 604/253

(57) ABSTRACT

An exemplary IV system can be provided which can include a drip chamber having a movable arrangement configured to mechanically move due to an impact thereof by at least one drop. The movable arrangement can include a spinning arrangement which is configured to spin due to the impact. The spinning arrangement can include a center portion having a plurality of protrusions, and can be mounted to the drip chamber using a mounting mechanism.

### CROSS-REFERENCE TO RELATED APPLICATION(S)

This application relates to and claims priority from U.S. patent application Ser. No. 61,697,423, filed on Sep. 6, 2012, the entire disclosure of which is incorporated herein by reference.

### FIELD OF THE DISCLOSURE

The present disclosure relates generally to an intravenous (“IV”) device, and more specifically, to an IV having a movable arrangement (e.g., a spinning wheel) to facilitate an easy detection of a flow of fluid inside the IV device.

### BACKGROUND INFORMATION

Current intravenous devices/apparatus can consist of an IV bag, which can house the IV fluid, and two lengths of a clear tubing. The top length of the tubing can be connected to the bottom length of the tubing via a bulbous coupling. The bulbous coupling can slow down the flow of fluid from the bag/top tubing into a steady drip rate appropriate for IV fluid administration. The bottom length of the tubing facilitates the calibrated IV drip to flow into the patient.

As the IV apparatus and the fluid can be both colorless and clear, it can be difficult to visualize the drip in the bulbous coupling of the IV device. It can also be difficult to determine whether or not the IV drip is flowing and at what rate. This can be problematic for both the nursing staff, who can be required to monitor IV flow, as well as the patient, who benefits from an assurance that the IV device and the drip are working properly (and who are often instructed by nursing staff to “make sure it is flowing”). The IV device in which the flow is stopped can occur due to blood clotting in the tube, an empty fluid bag, or a mechanical problem (e.g., a needle or catheter against the wall of the vein in which the needle has been inserted into). In such an event, manual cleaning may need to be performed, or in a worst-case scenario, another IV line must be established. Both such cases can require additional work by the nursing staff that can already be overburdened. Further, the additional work can add to the patient's anxiety, which can already be high by being in a hospital. If another IV lined needs to be established, it can require the removal of the present line, and the insertion of a new line by inserting a needle or catheter into a vein. This can be painful, and it can be difficult to find a new vein.

For example, FIG. 1 shows a conventional IV system 100. Such IV system 100 can include a drip chamber 40. Drip chamber 40 can be connected on a first side to first tubing 50, which can be connected to a supply bag (not shown), which can hold a medicament, saline, and/or other nutrients. Drip chamber 40 can be connected to second tubing 60 on a second side, which can be inserted into a patient through a needle or a cannula. As a liquid enters drip chamber 40, through tubing 50, liquid drops 70 descend from a first side of drip chamber 40 to a second side thereof, and exit drip chamber 40 into tubing 60. Typically, both drip chamber 40 and liquid drops 70 can be clear, and thus, it can be difficult to determine if the liquid is flowing, and if it is, at what rate.

Prior art systems have been developed to overcome some of the problems above. For example, International Patent Publication WO2009/114115 describes an IV device having different sensors, such as an impedance sensor, an electromechanical sensor, an optical sensor, a transmission sensor etc. U.S. Patent Publication No. 2008/0051732 describes an LED in conjunction with an optical sensor in order to determine if a drop of the fluid in the IV device passes through the LED and the optical sensor. A problem with these known devices is that they can be expensive to manufacture, and they can be easily susceptible to failure due to their complexity.

Thus, it may be beneficial to provide an exemplary IV arrangement/apparatus/device that can facilitate an easy detection of the flow of the fluid therein, that is non-complex and inexpensive to manufacture, and which can overcome at least some of the deficiencies described herein above.

### SUMMARY OF EXEMPLARY EMBODIMENTS

These and other objects of the exemplary embodiments of the present disclosure can be achieved by an exemplary IV coupler, which can include a valve arrangement, and a drip chamber connected to the valve arrangement. The drip chamber can include a movable arrangement, such as, for example, a spinning arrangement, which can be configured to move vertically, horizontally, in a uniform/non-uniform manner, spin, or move in any other manner based on a weight of a drop of liquid.

In certain exemplary embodiments of the present disclosure, the movable arrangement can include or be the spinning arrangement, which can comprise a center portion having a plurality of protrusions. The number of protrusions can be chosen to reduce or prevent a formation of a capillary bond(s), caused by the drop(s) of liquid between the protrusions. For example, the spinning arrangement can be mounted to a drip chamber using a mounting mechanism. The mounting mechanism can comprise two or more protrusions extending from a side of the drip chamber, which can contact the spinning arrangement and be configured to facilitate a rotation of the spinning arrangement. A distance between the moveable arrangement and the side of the drip chamber can be chosen based on a kinetic energy of the drop(s) of liquid when it is falling. In some exemplary embodiments of the present disclosure, the mounting mechanism can include a centering mechanism configured to keep the moveable arrangement substantially centered within the drip chamber. In certain exemplary embodiments of the present disclosure, a coefficient of friction between the moveable arrangement and the mounting mechanism can be selected to prevent the moveable arrangement from moving when no liquid is present within the drip chamber.

According to additional exemplary embodiments, the spinning arrangement can be shaped substantially similar to a water wheel. In one exemplary variant, the spinning arrangement can be made from a reflective material and/or a non-reactive material. Further, for example, the IV coupler can include a light source, which can include a light emitting diode. In some exemplary embodiments of the present disclosure, a valve arrangement can be configured to regulate a flow of the liquid in the drip chamber.

These and other objects, features and advantages of the exemplary embodiments of the present disclosure will become apparent upon reading the following detailed description of the exemplary embodiments of the present disclosure, when taken in conjunction with the appended exemplary claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is an example of a conventional IV system;

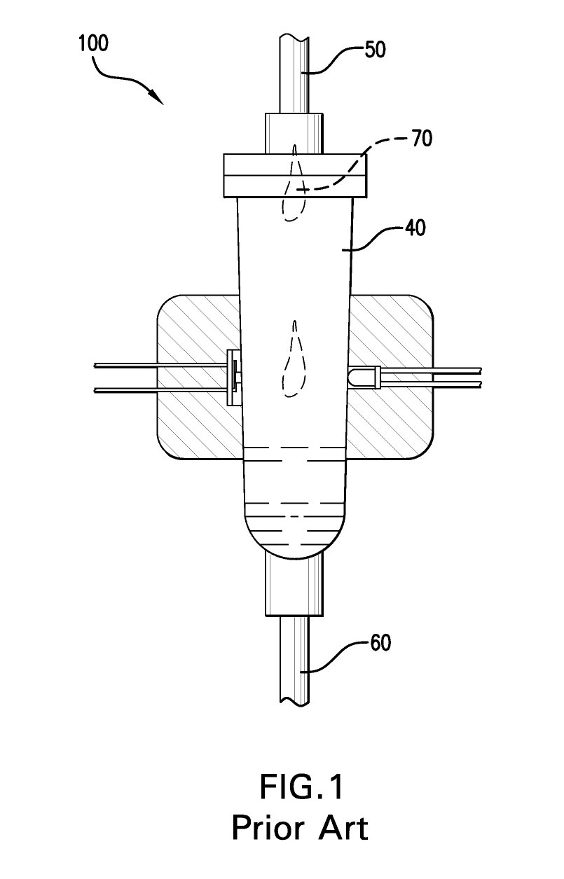

FIG. 2 is an exemplary IV system according to a first exemplary embodiment of the present disclosure;

FIG. 3 is the exemplary IV system having a different-shaped spinning arrangement according to a second exemplary embodiment of the present disclosure; and

FIG. 4 is the exemplary IV system having another different-shaped spinning arrangement according to an exemplary embodiment of the present disclosure.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures or the appended claims.

### DETAILED DESCRIPTION OF EXEMPLARY EMBODIMENTS

The exemplary embodiments of the present disclosure may be further understood with reference to the following description and the related appended drawings. The exemplary embodiments of the present disclosure relate to an exemplary IV system (e.g., device, apparatus, arrangement, system, etc.) to facilitate a non-complicated determination of the continuous flow of fluid, and the rate of flow of fluid, through the IV device. For example, the device can utilize a movable arrangement, such as and/or including, for example, a spinning wheel, in order to determine if the fluid is flowing in and/or through the IV system. The exemplary embodiments are described with reference to the IV device, although those having ordinary skill in the art will understand that the exemplary embodiments of the present disclosure can be implemented on any device where the easy detection of the flow of a liquid can be beneficial.

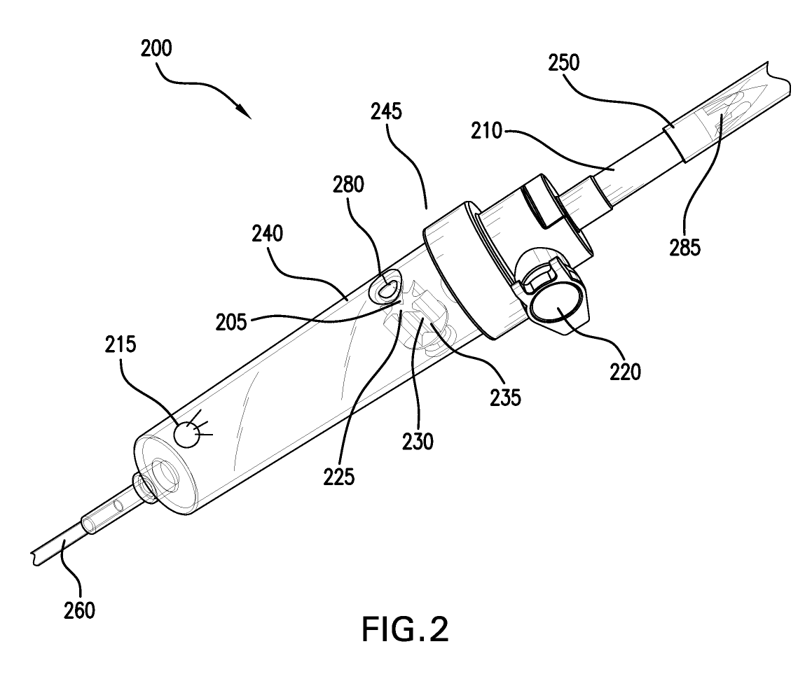

FIG. 2 shows an exemplary IV system 200 according to an exemplary embodiment of the present invention. Exemplary IV system 200 can include a coupling arrangement 245. Coupling arrangement 245 can include a drip chamber 240, and a valve arrangement 210. Valve arrangement 210 can be connected to a first tubing 250 via an end portion 285, which can have a shape of an “arrow head” in order to enter or be inserted into tubing 250. A second tubing 260 can be connected to drip chamber 240.

Valve arrangement 210 can include a valve or other liquid flow limiting device 220. Valve 220 can be used to adjust and/or control the flow of any liquid that passes through tubing 250, into valve arrangement 210, and then into drip chamber 240. The flow of the liquid can be adjusted and/or controlled to any suitable flow for use in exemplary IV system 200.

Drip chamber 240 can include a movable arrangement (e.g., including a spinning arrangement 230 such as a spinning wheel.) Spinning arrangement 230 can include a center portion 225 having, for example, any suitable shape, with a plurality of protrusions 235. Spinning arrangement 230 can include any suitable number of protrusions 235, however, the number of protrusions can be preferably small enough to prevent the formation of a capillary bond between the protrusions, which can increase the force needed to rotate spinning arrangement 230. Additionally, spinning arrangement 230 can have any suitable shape, such as a shape that can be substantially similar to that of a water wheel, and can be of any suitable size. In one exemplary embodiment, spinning arrangement 230 can be sized to be small enough to prevent a formation of a capillary bond between protrusions 235. The size of each individual drop, and its potential energy, can be modeled when designing the size and shape of the spinning arrangement 230. Additionally, the distance between the spinning arrangement 230 and a side of drip chamber 240, which contacts valve arrangement 210, can be modeled and/or modified based on the kinetic energy delivered by each drop at a specified distance. The distance can be chosen to overcome the friction and the inertia of spinning arrangement 230 and mounting mechanism 280.

Spinning arrangement 230 can have any suitable color, including alternating colors, and can be or have portions thereof that can be reflective and/or non-reflective, and can preferably be made from a non-reactive material. According to one exemplary embodiment of the present disclosure, spinning arrangement 230 can be made of any suitable material that can be light enough to facilitate the rotation of spinning arrangement 230 by the force of even a single liquid drop falling on spinning arrangement 230. In another exemplary embodiment, the material of spinning arrangement 230 can be heavy enough such that spinning arrangement 230 does not rotate when the fluid is not flowing in and/or through exemplary IV system 200.

Spinning arrangement 230 can be connected to drip chamber 240 via mounting mechanism 280. Mounting mechanism 280 can be formed in the sidewall of drip chamber 240 having two or more protrusions 205 which can contact spinning arrangement 230 and facilitate the rotation of spinning arrangement 230. Additional suitable mounting mechanisms can be employed. For example, mounting mechanism 280 can include a centering mechanism to aid in keeping spinning arrangement 230 centered, for example, so as to maintain the spinning ability of spinning arrangement 230. Additionally, the friction coefficient created between mounting mechanism 280 and spinning arrangement 230 can be sufficiently large such that spinning arrangement 230 does not rotate when there is no liquid flowing (e.g., by impacting it), but not so large as to prevent the rotation of spinning arrangement 230 by the liquid in exemplary IV system 200.

A light source, such as, for example, a light emitting diode (“LED”) 215, can be incorporated at any suitable location in coupling arrangement 245 to facilitate exemplary IV system 200 to be utilized in low light conditions.

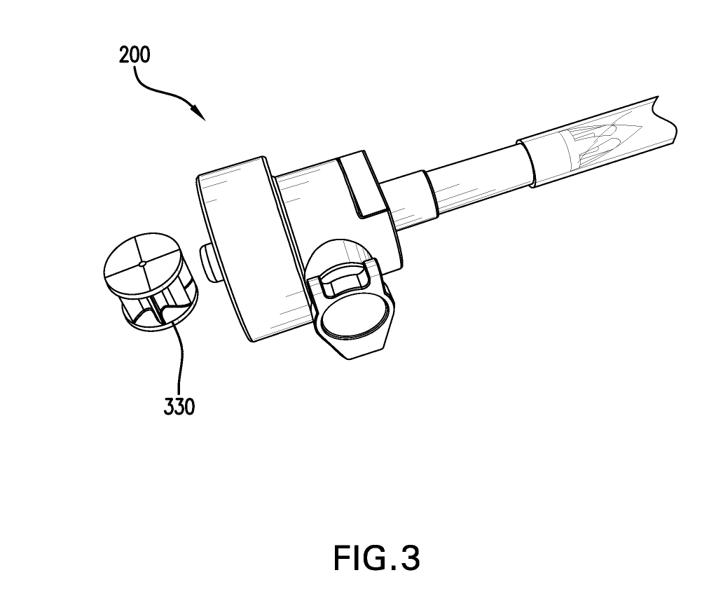

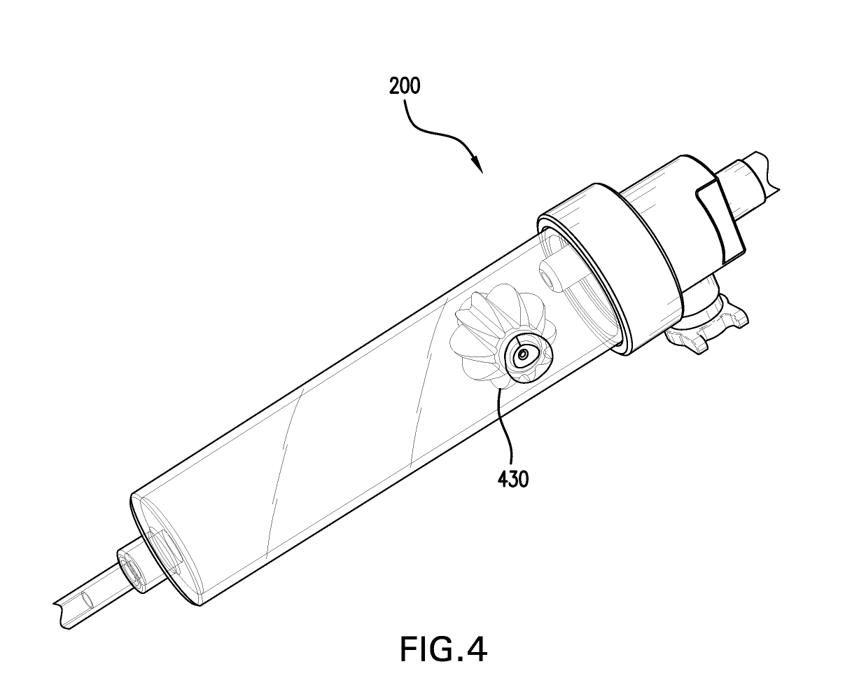

FIGS. 3 and 4 illustrate the exemplary IV system 200 having different shaped spinning arrangements 330 and 430, while using other similar or same components as those shown in the exemplary embodiment of FIG. 2.

Further, according to additional exemplary embodiments of the present disclosure, the movable arrangement can include arrangements other than spinning arrangements 230, 330, 430 (or in addition thereto) which can be configured to move vertically, horizontally, in uniform/non-uniform manner, spin, or move in any other manner based on a weight of a drop of liquid.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances. Further, to the extent that the prior art knowledge has not been explicitly incorporated by reference herein above, it is explicitly incorporated herein in its entirety. All publications referenced are incorporated herein by reference in their entireties.

What is claimed is:

1. An intravenous (IV) system, comprising:
a drip chamber comprising a movable arrangement configured to mechanically move due to an impact thereof by at least one drop of liquid.

2. The IV system of claim 1, wherein the movable arrangement comprises a spinning arrangement which is configured to spin due to the impact.

3. The IV system of claim 2, wherein the spinning arrangement comprises a center portion having a plurality of protrusions.

4. The IV system of claim 3, wherein a number of the protrusions is chosen to reduce or prevent a formation of at least one capillary bond, caused by the at least one drop of liquid, between the protrusions.

5. The IV system of claim 1, wherein the movable arrangement is mounted to the drip chamber using a mounting mechanism.

6. The IV system of claim 5, wherein the mounting mechanism comprises two protrusions extending from the side of the drip chamber and contacting the spinning arrangement and configured to allow for rotation of the spinning arrangement.

7. The IV system of claim 6, wherein a distance between the moveable arrangement and the side of the drip chamber is chosen based on a kinetic energy of the at least one drop of liquid when it is falling.

8. The IV system of claim 5, wherein the mounting mechanism includes a centering mechanism configured to keep the moveable arrangement substantially centered within the drip chamber.

9. The IV system of claim 5, wherein a coefficient of friction between the moveable arrangement and the mounting mechanism is selected to prevent the moveable arrangement from moving when no liquid is present within the drip chamber.

10. The IV system of claim 2, wherein the spinning arrangement is shaped substantially similar to a water wheel.

11. The IV system of claim 1, wherein the movable arrangement is made from a reflective material.

12. The IV system of claim 1, wherein the movable arrangement is made from a non-reactive material.

13. The IV system of claim 1, further comprising a light source.

14. The IV system of claim 13, wherein the light source is at least one light emitting diode.

15. The IV system of claim 1, wherein the movable arrangement comprises a plurality of fins.

16. The IV system of claim 15, wherein a first of the fins is composed of a reflective material and a second of the fins which is immediately adjacent to the first of the fins is composed of a non-reflective material.

17. The IV system of claim 15, wherein a first of the fins has a first color and a second of the fins which is immediately adjacent to the first of the fins has a second color.

18. The IV system of claim 17, wherein the first color is lighter than the second color.

19. The IV system of claim 1, further comprising a valve arrangement configured to regulate a flow of the liquid into the drip chamber.

20. The IV system of claim 2, wherein the spinning arrangement is made from at least one of a reflective material or a non-reactive material.
